(19) United States
(12) Patent Application Publication
(10) Pub. No.: US 2005/0065502 A1
(43) Pub. Date: Mar. 24, 2005

(54) Enabling or blocking the emission of an ablation beam based on color of target

(76) Inventor: Richard Stoltz, Plano, TX (US)

Correspondence Address: CHALKER FLORES, LLP, 12700 PARK CENTRAL, STE. 455, DALLAS, TX, 75251, US

(21) Appl. No.: 10/849,587

(22) Filed: May 19, 2004

Related U.S. Application Data
(60) Provisional application No. 60/494,172, filed on Aug. 11, 2003.
(60) Provisional application No. 60/503,578, filed on Sep. 17, 2003.

(51) Int. Cl. A61B 18/20

(52) U.S. Cl. 606/9; 606/10

(57) ABSTRACT

The present invention includes a method and apparatus for the surgical material removal from a body by optical-ablation by setting one or more color specifications for ablation control into a beam control system, measuring the color emitted from a portion of a surface in line with the beam path and selectively emitting or blocking the ablation beam when at least one measured color is within the one or more color specification for ablation control.

### CROSS-REFERENCE TO RELATED APPLICATIONS

This application claims priority to U.S. Provisional Patent Applications, Ser. No. 60/494,172; entitled “Enabling Or Blocking The Emission Of An Ablation Beam Based On Color Of Target Area,” filed Aug. 11, 2003 (Docket No. ABI-16); and Ser. No. 60/503,578 “Controlling Optically-Pumped Optical Pulse Amplifiers,” filed Sep. 17, 2003 (Docket No. ABI-23).

### TECHNICAL FIELD OF THE INVENTION

The present invention relates in general to the field of light amplification and, more particularly to controlling the emission of an ablation beam based on color of target area.

### BACKGROUND OF THE INVENTION

Ablative material removal is especially useful for medical purposes, either in-vivo or on the outside surface (e.g., skin or tooth). Ablative removal of material is generally done with a short optical pulse that is stretched amplified and then compressed. A number of types of laser amplifiers have been used for the amplification. Ablative material removal previously has been preformed using systems with optical benches weighing perhaps 1,000 pounds and occupying about 300 cubic feet.

Laser machining can remove ablatively material by disassociate the surface atoms and melting the material. Laser ablation is efficiently done with a beam of short pulses (generally a pulse-duration of three picoseconds or less). Techniques for generating these ultra-short pulses (USP) are described, e.g., in a book entitled “Femtosecond Laser Pulses” (C. Rulliere, editor), published 1998, Springer-Verlag Berlin Heidelberg New York. Generally large systems, such as Ti:Sapphire, are used for generating ultra-short pulses (USP).

USP phenomenon was first observed in the 1970's, when it was discovered that mode-locking a broad-spectrum laser could produce ultra-short pulses. The minimum pulse duration attainable is limited by the bandwidth of the gain medium, which is inversely proportional to this minimal or Fourier-transform-limited pulse duration. Mode-locked pulses are typically very short and will spread (i.e., undergo temporal dispersion) as they traverse any medium. Subsequent pulse-compression techniques are often used to obtain USP's. Pulse dispersion can occur within the laser cavity so that compression techniques are sometimes added intra-cavity. When high-power pulses are desired, they are intentionally lengthened before amplification to avoid internal component optical damage. This is referred to as “Chirped Pulse Amplification” (CPA). The pulse is subsequently compressed to obtain a high peak power (pulse-energy amplification and pulse-duration compression).

### SUMMARY OF THE INVENTION

Ablative material removal with short optical pulses is especially useful for medical purposes, as it is almost non-thermal and generally painless, and can be done either “in-vivo” or on a body surface. The present invention provides for enabling or blocking the emission of an ablation beam based on one or more colors of the area in line with the ablation beam for safety or ablation control reasons. This ablation as a function of the color of the target, can give the effect of a smart scalpel.

One embodiment, includes the selectively enabling or blocking of ablation by color. Another embodiment, includes the ablation by one color and blocking ablation by another color. Although the system scans over a larger area, the color-enabled system only uses ablation to remove certain areas, e.g., a tattoo one color at a time, or a mole. In one embodiment, a video camera, having separate internal signals for red, green, and blue, is used to determine feedback and determine where to ablate. In one embodiment, a color is selected form a set of colors displayed on a computer monitor using a computer mouse and the color specification is loaded into the control system. One embodiment, uses a control system can determine ratios (e.g., of Blue/Green and Red/Green) to control the emitting or blocking of the ablation on appropriate areas of the surface depending on the ratios. One embodiment, allows ranges of color ratios to be specified (e.g., narrow range, normal range, or wide range). Thus, the control inputs of a range of two (2) ratios (e.g., of B/G and R/G) can be set to define the color. The color of the reflection is influenced by the illumination spectrum. In another embodiment, a black-and-white video camera is used with a control input based wide bandwidth intensity (brightness) controlling contrast.

Some embodiments allow the ablation probe to be a handheld probe, while other embodiments allow the ablation probe is mounted on an x-y or x-y-z-positioner. The mounting of the ablation probe mounted on an x-y-z-positioner allows the probe to be moved in the z-direction to follow surfaces that are not flat. In other embodiments smaller ablation areas can be scanned through moving the beam without moving the probe. In one embodiment, large areas may be scanned by moving the beam over a first area with the probe position essentially stationary, and then stepping the probe to second portion of the large area and then scanning the beam over the second area, and so on.

One embodiment, allows put colored marker (e.g., a blue or white cream) to be placed on the skin where ablate is not to be preformed (the cream could also shield skin from unwanted ablation). Other embodiments use a UV illumination and a UV camera, or an IR illumination and an IR camera. Other embodiments use an IR camera to sense temperature of bodies at or above ambient temperature, e.g., tumors. As used herein, the term “light” includes photons of wavelengths from UV, through the visible, and through the IR, and the term “color” includes relations of wavelengths in the V, visible, and IR, and also contrasts in intensity of reflections between one surface area and another surface area. Another embodiment, includes a combination IR and visible camera to provide alignment between the ablation beam and the camera. The IR camera (or IR portion of a dual IR-visible camera) are sensitive to small temperature change caused by the ablation, e.g., place a beam-marker on the video display.

In one embodiment, a user illuminates (e.g., a laser-pointer) the area to be ablate. In some embodiments the probe is mounted on an x-y or x-y-z-positioner. The user then direct ablation to the illuminated spot, thus, reducing accidental injuries or damage.

One embodiment, of the present invention includes a method of controlling an ablation beam emitted in a beam path from a probe, including setting one or more at color specification for ablation control into a beam control system; measuring colors emitted from a portion of a surface in line with the beam path; and selectively emitting or blocking the ablation beam when one or more measured colors are within at least one the color specification for ablation control.

In one embodiment, the beam is amplified in an optically-pumped amplifier and the blocking is accomplished by shutting off the current to the optically-pumped-amplifiers pump diodes. In another embodiment, the beam is amplified in a semiconductor optical amplifier and the blocking is accomplished by shutting off the current to the semiconductor optical amplifier. Other embodiments enable the beam through supplying current to the amplifier. In another embodiment, the blocking is accomplished through measuring a color within a blocking color-specification for ablation control. The beam is emitted when the measured color is not within the blocking color-specification for ablation control, and is within a emitting color-specification for ablation control. The color-spectrum can include one or more colors. In other embodiments, the measured color may also be either of IR colors or of UV colors. Another embodiment includes an over-ride switch that allows ablation to be enabled when a measured color is within the blocking color-specification for ablation control. Other embodiments may include a user controlled on/off button or switch. Other embodiments include an auxiliary light beam that changes color or an audible signal, when the beam is on.

One embodiment, of the present invention includes a method of controlling the emission of an ablation beam in a beam path from a probe, including setting one or more color specification for ablation control into a beam control system; measuring color emitted from a portion of a surface in line with the beam path; and emitting or blocking the ablation beam when at least one measured color is within the one or more color specification for ablation control.

### DETAILED DESCRIPTION OF THE INVENTION

While the making and using of various embodiments of the present invention are discussed in detail below, it should be appreciated that the present invention provides many applicable inventive concepts that can be embodied in a wide variety of specific contexts. The specific embodiments discussed herein are merely illustrative of specific ways to make and use the invention and do not delimit the scope of the invention.

To facilitate the understanding of this invention, a number of terms are defined below. Terms defined herein have meanings as commonly understood by a person of ordinary skill in the areas relevant to the present invention. Terms such as “a”, “an” and “the” are not intended to refer to only a singular entity, but include the general class of which a specific example may be used for illustration. The terminology herein is used to describe specific embodiments of the invention, but their usage does not delimit the invention, except as outlined in the claims.

As used herein, the term “light” includes photons of wavelengths from UV, through the visible, and through the IR, and the term “color” includes relations of wavelengths in the UV, visible, and IR, and also contrasts in intensity of reflections between one surface area and another surface area.

As used herein, human vision and video cameras both sense color information by light wavelength, sensing blue at wavelengths around 450 mn, green at wavelengths around 540 mn, and red at wavelengths around 610 nm, and other colors are recognized by the brain by comparing intensities of the three regions (the eye has three response curves, one peaking at each of these three wavelengths).

Ablative material removal with short optical pulses is especially useful for medical purposes. One embodiment of the present invention includes a method for enabling or blocking the emission of an ablation beam based on the color of the area in line with the ablation beam for safety or ablation control reasons. In one embodiment, the system scans over an area, wherein the color-enabled system only ablates to remove certain portions of that area, e.g., a tattoo (one or more colors at a time) or a mole. In another embodiment, the emission is blocked where a colored cream is present on the skin, thus, indicating the area the user does not want to ablate (the cream could also shield skin from unwanted ablation). In some embodiments, the colored cream may be blue, white, green, yellow or other desired color or combination of colors. In other embodiments, a laser pointer is used to indicate the region for ablation. In one embodiment, the ablation and/or blocking ablation is through the reflection of color, e.g., with 3-color or broadband-white illumination or with illumination that enhances color contrast (see, e.g., Thornton U.S. Pat. No. 3,963,953). In another embodiment, ablation is conducted on tumors based on the higher-temperature-indicating infrared light emitted by the tumor. This ablation as a function of the color of the target, therefore gives the effect of a smart scalpel.

Ablative material removal previously has been done using systems with optical benches weighing perhaps 1,000 pounds and occupying about 300 cubic feet. One embodiment of the present invention includes an ablative system that can weigh 100 pounds or less and occupy less than 2.5 cubic feet. One embodiment includes multiple moderate-power semiconductor-optical-amplifiers or fiber amplifiers, with a short initial optical pulse that undergoes controlled amplification and is then compressed into a short pulse, and the light pulse focused onto a small area spot. One embodiment rapidly scans the spot over an area to be ablated and produces a controllable rate of ablation with the small spot. One embodiment of the present invention controls the amplifiers to give a pulse power controlled for optimum ablation efficiency. The concentration of pulse energy on a small spot enables the use of semiconductor-optical amplifiers or moderate-power optically-pumped amplifiers. The use a short initial optical pulse allows compression into a short pulse with an efficient and physically small compressor. The use of multiple moderate-power amplifiers allows ablation rate and pulse energy to be independently controlled. Additionally, one embodiment of the present invention allows the use of easily cooled amplifiers. Thus, by the use of a combination of innovations, can now provide an efficient, reasonably-priced, man-portable ablation system (e.g., a wheeled cart or a backpack) for medical and other purposes.

One embodiment of the present invention includes the enabling or blocking of ablation by color (or ablation by one color and blocking ablation by another color). In one embodiment, a video camera is used to obtain feedback indicating an area to ablate. Generally, a video camera (or RGB computer monitor) has internal separate signals for Red, Green, and Blue. In one embodiment, the color is selected on the monitor. In one embodiment, a computer mouse may be used to select a color on a monitor to set the color specification into the control system. In one embodiment, the control system can determine the color ratios (e.g., of Blue/Green and Red/Green) and then emit or block, the ablation on the appropriate areas of the surface depending on the ratios. In one embodiment, a range of color ratios (e.g., narrow range, normal range, or wide range) are specified. In one embodiment, the control inputs of a range of two (2) ratios (e.g., of Blue/Green and Red/Green) is set to define the color. Other embodiments can use different ranges of ratios and differing ratios to define the color. The color of the reflection is influenced by the illumination spectrum. In another embodiment, a control input is based total intensity (brightness), e.g., controlling on contract and using a black-and-white video camera

In one embodiment, a camera is used “in-vivo” (see “Camera Containing Medical Tool” provisional application No. 60/472,071; Docket No. ABI-4; filed May 20, 2003; which is incorporated by reference herein) including an optical fiber in a probe to convey an image to a camera. In one embodiment, the camera is a vidicon-containing remote camera body. One embodiment uses a handheld probe which can supply its own illumination. Another embodiment of the present invention uses an optical fiber in a probe to convey an image back to a remote camera with a GRIN fiber lens. Yet another embodiment uses an endoscope type camera.

In some embodiments, the ablation probe is a handheld probe, and in other embodiments the ablation probe is mounted on an x-y or x-y-z-positioner. In one embodiment, the control system position controls movement in the x, y, and z directions, independently and/or in combination. One embodiment of the present includes a laser-pointer to direct ablation and a tracking system that tracks laser-pointer. In one embodiment, the probe is mounted on a control system positionable table. In one embodiment, the tracking system includes five sensors in a cross configuration (which may be remote from the probe) that receive and convey reflections via optical fiber in the probe, to automatically compare the signals from the five sensors and to position the probe such that the ablation beam is pointed at the area monitored by the center sensor. In one embodiment, the sensors is an optical fiber in a probe to convey an image back to a remote sensor. In one embodiment, the sensor is a vidicon-containing remote camera body as in the above “Camera Containing Medical Tool” provisional application. Another embodiment uses a light detector sensitive to the laser-pointer wavelength.

One embodiment of the present invention combines an IR and visible camera to provide alignment between the ablation beam and the camera. The IR camera (or IR portion of a dual IR-visible camera) senses small temperature change caused by the ablation and displays a beam-marker on the video display. In one embodiment, the beam-marker is of a different color than the laser-pointer. In one embodiment, the combination of color and time is used for evaluation after a prescribed time.

In one embodiment, the ablation probe is mounted on an x-y-z-positioner, and the probe moved in the z-direction to follow surfaces that are not flat. In another embodiment, the system automatically adjusts the output optics to focus the beam at the appropriate distance to follow surfaces that are not flat. One embodiment scans the ablation areas by moving the beam without moving the probe. One embodiment scans a larger area by moving the beam over a first area, and then stepping the probe to second portion of the large area and then scanning the beam over the second area, and so on. In other embodiments the scanning is accomplished using beam deflecting mirrors mounted on piezoelectric actuators (see “Scanned Small Spot Ablation With A High-Rep-Rate” U.S. Provisional Patent Applications Ser. No. 60/471,972, Docket No. ABI-6; filed May 20, 2003; which is incorporated by reference herein).

In some embodiments, the predefined target parameters are used to specify the area to be ablated. One embodiment of the present invention scans an area with the ablation beam only enabled to ablate portions within a defined color and/or area. Yet other embodiments scan the area positioning the beam only over a defined color. In one embodiment, the system allows ablation pulses only within predefined target parameters. In some embodiments the predefined target parameters are a range of probe to target distances based on measurements. Types of measurements can be made by sonic feedback, or size of conical or cross-shaped auxiliary light beam with on target, or from backpressure of an air-jet (or air-jet and suction combination), or other methods know in the art.

In one embodiment, a marker is placed on the surface of an object to allow the system to correct for movement. In addition to external surfaces, internal surfaces can be analyzed for color and ablated. In one embodiment internal surfaces are analyzed and ablated through controlling the fluid in the beam path. In one embodiment an endoscope and ablation probe are inserted into a body (again see “Camera Containing Medical Tool” provisional application No. 60/472,071).

In one embodiment, the beam is amplified in a fiber amplifier and the blocking is accomplished by shutting off the current to the fiber-amplifiers pump diodes. In another embodiment, the beam is amplified in a semiconductor optical amplifier and the blocking is accomplished by shutting off the current to the semiconductor optical amplifier. In another embodiment blocking is accomplished by the insertion of an adsorbing material in the beam path. However, those skilled in the art will recognize that other method of blocking could also be used.

In one embodiment, controlling the pump diode current controls the temperature of a fiber-amplifier. In another embodiment adjusting the repetition rate of the pulse generator controls the pulse energy. In another embodiment two or more amplifiers are used in a train mode (pulses from one amplifier being delayed to arrive one or more nanoseconds after those from another amplifier) allowing step-wise control of ablation rate independent of pulse energy. One embodiment of the present invention has pulses approximately three times the ablation threshold, whereby greater ablation efficiency is achieved.

In one embodiment the emission of an ablation beam is enabled or blocked based on distance to the surface. In one embodiment, the distance from the surface to be ablated is measured sonically (measuring time between “ping” and receiving of the echo, much like sonar). In another embodiment, the distance is measured is by measuring backpressure from air-jet (or suction induced pressure, or air-jet and suction combination) as a nearby object slows the flow, which raises the jet backpressure (or drop the pressure in the suction line). In one embodiment, the beam can be blocked if no signal indicating a distance less than the maximum distance is received (see “Altering The Emission Of An Ablation Beam for Safety or Control” provisional application Docket No. ABI-15; which is incorporated by reference herein). In another embodiment, a similar distance control system repositions the probe to maintain the probe in a predetermined range of distance from the surface being ablated.

In another embodiment, the distance is measured by measuring a dimension of size of an auxiliary light beam with on an object and blocking the beam if no signal indicating a distance less than the maximum distance is received. In one embodiment, the measurement is sensed with a video camera and the video signal monitored (e.g., for the longest of times in which color of the auxiliary beam remains in a video scan). In another embodiment an auxiliary light beam is used to indicate to a user the region to be ablated. In one embodiment, the auxiliary light beam is conical or has a line or cross shape. In one embodiment, the auxiliary light beam changes color when the beam is on. In other embodiments the auxiliary light beam may be scanned. In one embodiment, the beam scan length and auxiliary light-beam length can be varied as a function of the distance from the object, whereby the length can be displayed before the ablation takes place and then the ablation controlled to give that length. The area of ablation can be similarly displayed and controlled.

In one embodiment, the ablation threshold is less than one Joule per square centimeter. Other embodiments have an ablation threshold of up to about two Joules per square centimeter. In one embodiment, the ablation rate is controlled independent of pulse energy. In one embodiment, two or more amplifiers are used in a train mode (pulses from one amplifier being delayed to arrive one or more nanoseconds after those from another amplifier) allowing a step-wise control of ablation rate independent of pulse energy. One embodiment desiring a lower ablation rates, controls pulses by shutting off one or more amplifiers (e.g., the optical pumping to the fiber amplifier shut off). For example 20 amplifiers there would be a maximum of 20 pulses in a train. In other embodiment three or four amplifiers are used producing three or four pulses per train.

Generally, the optically-pumped amplifiers are optically-pumped continuous wave (CW) or quasi-CW (pumping and amplifying perhaps 500 times per second in one millisecond bursts). In quasi-CW, there is a pause between bursts and the ratio of durations of the pause and the burst may be adjusted for component temperature and/or average repetition rate control. Additionally, amplifiers may be run in a staggered fashion, e.g., one on for a first half-second period and then turned off for a second half-second period, and another amplifier, dormant during the first-period, turned on during the second period, and so forth, to spread the heat load.

One embodiment use sub-picosecond pulses of between ten picoseconds and one nanosecond, followed by pulse selection, with the selected pulses amplified by a fiber-amplifier (e.g., a erbium-doped fiber amplifier or EDFA) and compressed by an air-path between gratings compressor (e.g., a Tracey grating compressor), with the compression creating a sub-picosecond ablation pulse. In one embodiment, a semiconductor oscillator is used to generate pulses and in another embodiments a SOA preamplifier is used to amplify the selected pulses before introduction into the fiber amplifier.

Generally, compressors can be run with inputs from more than one amplifier, reflections from other of the parallel amplifiers can cause a loss of efficiency, and thus should be minimized. The loss is especially important if the amplifiers are amplifying signals at the same time, as is the case with the SOAs. In one embodiment each parallel SOAs has its own compressor and the amplified pulses are put into a single fiber after the compressors, thus, reflections from the joining (e.g., in a star connector) are reduced greatly. In one embodiment two or more fiber amplifiers, with a nanosecond spacing of sub-nanosecond pulses, uses a single compressor.

Fiber amplifiers have a storage lifetime of about 100 to 300 microseconds. Some fiber amplifiers measurements have been made at higher rep rates (and these measurements have shown an approximately linear decrease in pulse energy). In one embodiment fiber amplifiers for ablation have been operated with a time between pulses of about equal to or greater than the storage lifetime, and thus are generally run a rep rate of less than 3-10 kHz.

Amplifiers are available with average power of 30 W or more. In one embodiment, a moderate-power 5 W average power fiber amplifiers is operated to give pulses of 500 microjoules or more, as energy densities above the ablation threshold are needed for non-thermal ablation, and increasing the energy in such a system, increases the ablation rate in either depth or allows larger areas of ablation or both. One embodiment produces a small ablation spot using a fiber amplifier with a time between pulses of a fraction (e.g., one-half or less) of the storage lifetime. In one embodiment, the ablation spot is less than approximately 50 microns in diameter. In one embodiment, the beam is scanned to produce a larger effective ablation area.

One embodiment uses a one ns pulse with a fiber amplifier and air optical-compressor (e.g., a Tracey grating compressor) typically gives compression with ˜40% losses. At less than one ns, the losses in a Tracey grating compressor are generally lower. If the other-than-compression losses are 10%, two nanoJoules are needed from the amplifier to get one nanoJoule on the target. One embodiment uses 1550 nm light. The use of greater than one ns pulses in an air optical-compressor presents two problems; the difference in path length for the extremes of long and short wavelengths needs to be more three cm and thus the compressor is large and expensive, and the losses increase with a greater degree of compression.

One embodiment increases the ablation rate using parallel fiber amplifiers generate a train of pulses to increase effective repetition rate. In one embodiment control of the number of operating fiber amplifiers is used to avoid thermal problems. One embodiment uses a SOA preamplifier to amplify the initial pulse before splitting to drive multiple parallel fiber amplifiers and another SOA before the introduction of the signal into each fiber amplifier, thus allowing rapid shutting down of individual fiber amplifiers.

One embodiment uses a semiconductor-oscillator to generate an initial pulse as part of a pulse generator that controls the repetition rate input into the amplifier. One embodiment uses one or more SOA preamplifiers to pre-amplify the pulse. In one embodiment parallel amplifiers are used to generate a train of pulses to increase the ablation rate by further increasing the effective repetition rate (while avoiding thermal problems and allowing control of ablation rate by adjusting the number of operating amplifiers).

In one embodiment, the input optical signal power, optical pumping power of fiber amplifiers, timing of input pulses, length of input pulses, and timing between start of optical pumping and start of optical signals to control pulse power, and average degree of energy storage in fiber can be controlled. In one embodiment, the pulse generator controls the input repetition rate of the fiber amplifiers to tune energy per pulse to approximately three times threshold per pulse.

In one embodiment, an initial current-swept-with-time electrical pulse is generated and then impressed on a laser diode to produce an optical wavelength-swept-with-time pulse. Another embodiment generates a sub-picosecond pulse and time-stretching that pulse within semiconductor pulse generator to give the initial wavelength-swept-with-time initial pulse.

One embodiment of the present invention measures light leakage from the delivery fiber to produce feedback proportional to pulse power and/or energy for control purposes. One embodiment measures spot size of a stationary spot using a video camera, another embodiment measures spot size with a linear scan.

One embodiment uses a camera “in-vivo” type (see “Camera Containing Medical Tool” provisional application No. 60/472,071; Docket No. ABI-4; filed May 20, 2003; which is incorporated by reference herein) using an optical fiber in a probe to convey an image back to a vidicon-containing remote camera body. One embodiment uses a handheld beam-emitting probe.

One embodiment scans smaller ablation areas by moving the beam without moving the probe. Other embodiments scan a larger area by moving the beam over a first area, and then stepping the probe to second portion of the large area and then scanning the beam over the second area, and so on. In one embodiment, the scanning is accomplished using beam deflecting mirrors mounted on piezoelectric actuators (see “Scanned Small Spot Ablation With A High-Rep-Rate” U.S. Provisional Patent Applications Ser. No. 60/471,972, Docket No. ABI-6; filed May 20, 2003; which is incorporated by reference herein). In one embodiment, the actuators scan over a large region, wherein the ablation beam is only enabled to ablate portions with defined color and/or area. One embodiment allows evaluation after a prescribed time by presetting a combination of time and, area and/or color.

One embodiment of the present invention combines a fiber-amplifier and a small pulse-compressor enabling a practical and significant size reduction, which in turn enables the system to be man-portable, e.g., as a wheeled cart and/or even in a backpack. As used herein, the term “man-portable” means capable of being moved reasonably easily by one person, e.g., as wheeling a wheeled cart from room to room or being carried in a backpack. In one embodiment of the present invention the system is man-portable including a cart and/or a backpack, and a probe. One embodiment includes an audible and/or visible indication (e.g., color change of the auxiliary beam) of when ablation pulse is active.

Information of such a system and other information on ablation systems are given in co-pending provisional applications listed in the following paragraphs (which are also at least partially co-owned by, or exclusively licensed to, the owners hereof) and are hereby incorporated by reference herein (provisional applications listed by Docket number, title and U.S. Provisional Patent Applications Ser. No.):
Docket No. ABI-1 “Laser Machining” U.S. Provisional Patent Applications Ser. No. 60/471,922; Docket No. ABI-4 “Camera Containing Medical Tool” U.S. Provisional Patent Applications Ser. No. 60/472,071; Docket No. ABI-6 “Scanned Small Spot Ablation With A High-Rep-Rate” U.S. Provisional Patent Applications Ser. No. 60/471,972; and Docket No. ABI-7 “Stretched Optical Pulse Amplification and Compression”, U.S. Provisional Patent Applications Ser. No. 60/471,971, were filed May 20, 2003;
Docket No. ABI-8 “Controlling Repetition Rate Of Fiber Amplifier” U.S. Provisional Patent Applications Ser. No. 60/494,102; ABI-9 “Controlling Pulse Energy Of A Fiber Amplifier By Controlling Pump Diode Current” U.S. Provisional Patent Applications Ser. No. 60/494,275; ABI-10 “Pulse Energy Adjustment For Changes In Ablation Spot Size” U.S. Provisional Patent Applications Ser. No. 60/49,4274; Docket No. ABI-11 “Ablative Material Removal With A Preset Removal Rate or Volume or Depth” U.S. Provisional Patent Applications Ser. No. 60/494,273; Docket No. ABI-12“Fiber Amplifier With A Time Between Pulses Of A Fraction Of The Storage Lifetime”; Docket No. ABI-13 “Man-Portable Optical Ablation System” U.S. Provisional Patent Applications Ser. No. 60/494,321; Docket No. ABI-14 “Controlling Temperature Of A Fiber Amplifier By Controlling Pump Diode Current” U.S. Provisional Patent Applications Ser. No. 60/494,322; Docket No. ABI-15 “Altering The Emission Of An Ablation Beam for Safety or Control” U.S. Provisional Patent Applications Ser. No. 60/494,267; Docket No. ABI-17 “Remotely-Controlled Ablation of Surfaces” U.S. Provisional Patent Applications Ser. No. 60/494,276 and Docket No. ABI-18 “Ablation Of A Custom Shaped Area” U.S. Provisional Patent Applications, Ser. No. 60/494,180; were filed Aug. 11, 2003. Docket No. ABI-19 “High-Power-Optical-Amplifier Using A Number Of Spaced, Thin Slabs” States Provisional Patent Applications Ser. No. 60/497,404 was filed Aug. 22, 2003;
Co-owned Docket No. ABI-20 “Spiral-Laser On-A-Disc”, U.S. Provisional Patent Applications Ser. No. 60/502,879; and partially co-owned Docket No. ABI-21 “Laser Beam Propagation in Air”, U.S. Provisional Patent Applications Ser. No. 60/502,886 were filed on Sep. 12, 2003. Docket No. ABI-22 “Active Optical Compressor” U.S. Provisional Patent Applications Ser. No. 60/503,659, filed Sep. 17, 2003;
Docket No. ABI-24 “High Power SuperMode Laser Amplifier” U.S. Provisional Patent Applications Ser. No. 60/505,968 was filed Sep. 25, 2003, Docket No. ABI-25 “Semiconductor Manufacturing Using Optical Ablation” U.S. Provisional Patent Applications Ser. No. 60/508,136 was filed Oct. 2, 2003, Docket No. ABI-26 “Composite Cutting With Optical Ablation Technique” U.S. Provisional Patent Applications Ser. No. 60/510,855 was filed Oct. 14, 2003 and Docket No. ABI-27 “Material Composition Analysis Using Optical Ablation”, U.S. Provisional Patent Applications Ser. No. 60/512,807 was filed Oct. 20, 2003; Docket No. ABI-28 “Quasi-Continuous Current in Optical Pulse Amplifier Systems” U.S. Provisional Patent Applications Ser. No. 60/529,425 and Docket No. ABI-29 “Optical Pulse Stretching and Compressing” U.S. Provisional Patent Applications Ser. No. 60/529,443, were both filed Dec. 12, 2003;
Docket No. ABI-30 “Start-up Timing for Optical Ablation System” U.S. Provisional Patent Applications Ser. No. 60/539,026; Docket No. ABI-31 “High-Frequency Ring Oscillator”, U.S. Provisional Patent Applications Ser. No. 60/539,024; and Docket No. ABI-32 “Amplifying of High Energy Laser Pulses”, U.S. Provisional Patent Applications Ser. No. 60/539,025; were filed Jan. 23, 2004; and
Docket No. ABI-33 “Semiconductor-Type Processing for Solid-State Lasers,” U.S. Provisional Patent Applications Ser. No. 60/543,086, was filed Feb. 9, 2004; and Docket No. ABI-34 “Pulse Streaming of Optically-Pumped Amplifiers”, U.S. Provisional Patent Applications Ser. No. 60/546,065, was filed Feb. 18, 2004. Docket No. ABI-35 “Pumping of Optically-Pumped Amplifiers,” was filed Feb. 26, 2004.

Although the present invention and its advantages have been described in detail, it should be understood that various changes, substitutions and alterations can be made herein without departing from the spirit and scope of the invention as defined by the appended claims. While use by a surgeon is an important use, the system can have other uses, and the optically-pumped amplifier, may be a Cr:YAG amplifier. Moreover, the scope of the present application is not intended to be limited to the particular embodiments of the process, machine, manufacture, composition of matter, means, methods and steps described in the specification, but only by the claims.

1. A method of controlling a surgical ablation beam emitting in a beam path from a surgical-probe, comprising:
setting one or more color specifications for ablation control into a beam control system;
measuring color emitted from a portion of a surface in line with the beam path; and
selectively emitting or blocking the ablation beam when one or more measured color is within the at least one color specification for ablation control.

2. The method of claim 1, wherein the beam is amplified in an optically-pumped amplifier and the blocking is by shutting off the current to the optically-pumped-amplifiers pump diodes or wherein the beam is amplified in a semiconductor optical amplifier and the blocking is by shutting off the current to the semiconductor optical amplifier.

3. The method of claim 1, wherein the beam is amplified in an optically-pumped amplifier and the enabling is by turning on the current to the optically-pumped-amplifiers pump diodes or wherein the beam is amplified in a semiconductor optical amplifier and the enabling is by turning on the current to the semiconductor optical amplifier.

4. The method of claim 1, wherein the blocking is when the measured color is within a blocking color specification for ablation control and the emitting is when the measured color is not within the blocking color specification for ablation control, but is within a emitting color specification for ablation control.

5. The method of claim 4, further comprising an over-ride switch, whereby ablation is controlled to be enabled when the measured color is within the first color specification for ablation control.

6. The method of claim 1, wherein the measured color is either an IR color or a UV color.

7. The method of claim 4, wherein a computer mouse is used to select a color on a computer monitor to set the color specification into the control system.

8. The method of claim 1, wherein there is a user controlled on/off button or switch.

9. The method of claim 1, wherein there is an audible signal or an auxiliary light beam changes color when the beam is on.

10. The method of claim 1, wherein the ablation-probe is handheld by the surgeon.

11. The method of claim 1, wherein the ablation-probe is mounted on a control-system-positionable table, and a handheld laser pointer is used by a surgeon to direct ablation.

12. A method of controlling an ablation beam emitting in a beam path from a probe, comprising:
one or more color specifications for ablation control into a beam control system;
measuring color emitted from a portion of a surface in line with the beam path; and
selectively emitting or blocking the ablation beam when one or more measured color is within the at least one color specification for ablation control.
